(19) United States
(12) Patent Application Publication
(10) Pub. No.: US 2025/0032814 A1
(43) Pub. Date: Jan. 30, 2025

(54) METHOD FOR RELIEVING PAIN IN SURFACE ELECTRODE-BASED RF THERAPY BY CONTROLLING OUTPUT ENERGY PER PULSE AND TIMING OF COOLING, AND DEVICE THEREFOR

(71) Applicant: SKINGRAB CO., LTD., Cheonan-si (KR)

(72) Inventor: Sugun LEE, Paju-si (KR)

(73) Assignee: SKINGRAB CO., LTD., Cheonan-si (KR)

(21) Appl. No.: 18/786,432

(22) Filed: Jul. 26, 2024

Related U.S. Application Data
(63) Continuation-in-part of application No. PCT/KR2023/019985, filed on Dec. 6, 2023.

(30) Foreign Application Priority Data
Jul. 28, 2023 (KR) 10-2023-0098634
Jun. 18, 2024 (KR) 10-2024-0078715

(51) Int. Cl. A61N 1/40

(52) U.S. Cl. CPC A61N 1/40

(57) ABSTRACT

Disclosed is a pain relief device for relieving pain through control of output energy for each pulse and timing of cooling during surface electrode-based RF therapy. The pain relief device includes an electrode part in contact with surface of skin, an energy supply part that supplies energy to the electrode part during a reference cycle, a cooling part, and a processor that controls the energy supply part and the cooling part. The pain relief device may achieve excellent skin treatment effects while reducing skin pain.

### CROSS-REFERENCE TO RELATED APPLICATIONS

The present application is a continuation-in-part application of International Patent Application No. PCT/KR2023/019985, filed on Dec. 6, 2023, and claims the benefit of priority under 35 U.S.C. § 119 to Korean Patent Application Nos. 10-2023-0098634 filed on Jul. 28, 2023 and 10-2024-0078715 filed on Jun. 18, 2024 in the Korean Intellectual Property Office. The entire contents of the above applications are hereby incorporated by reference.

### BACKGROUND

Embodiments of the present disclosure described herein relate to techniques related to procedures for non-invasive skin treatment. More specifically, embodiments of the present disclosure relate to a method for relieving pain by controlling output energy per pulse and controlling the timing of cooling during non-contact, indirect contact, or capacitively coupled surface electrode-based RF therapy and a device therefor.

The following description merely provides background information related to embodiments and does not constitute prior art.

Skin care devices for removing wrinkles, restoring skin elasticity, removing sebum, and/or the like include a method of delivering ultrasonic waves to skin tissue (HIFU type), a method of delivering radio frequency (RF) waves to skin tissue (RF type), and a method of irradiating skin tissue with laser light (Optical type).

A conventional invasive device that delivers RF to skin tissue repeatedly invades the deep layers of skin (e.g., the dermis layer) with RF needle electrodes that reciprocate in a vertical direction, and uses the heat generated by radio frequencies to remove damaged collagen, elastic fibers, and/or the like from the deep layers of the skin and promote new formation. In addition, these skin care devices may help to improve the pigmentation of the skin or to remove acne marks and wrinkles.

However, unexpected damage may occur due to the skin invasion, and even with anesthesia, there may be pain afterward. Specifically, when the temperature of the skin tissue is excessively raised by RF energy, pain may be triggered by the stimulation of nociceptors by heat. In the case of high-power RF energy, the temperature of the epidermis and dermis may increase rapidly, which may cause persistent pain during and after the procedure. Even with anaesthesia, pain may still occur as long as the tissue damage or thermal irritation caused by RF energy persists to some extent. Therefore, there is a need for a non-invasive device for therapeutic (or cosmetic) treatment of the skin.

As described above, conventional RF therapy methods have caused significant pain and discomfort due to rapid temperature increases at the surface of the skin. In particular, when high power RF energy is used, the conventional RF therapy methods have caused rapid temperature increases in the epidermis and dermis of the skin, causing persistent pain during and after treatment. Furthermore, the conventional RF therapy methods have difficulty in delivering sufficient energy to the deep layers of the skin while simultaneously maintaining a safe surface temperature. Accordingly, the present disclosure seeks to address these problems and achieve both effective treatment and patient comfort.

### SUMMARY

Embodiments of the present disclosure provide a treatment device for skin treatment, which utilizes surface electrodes to deliver electrical energy to the deep layers of skin in a non-invasive manner.

Embodiments of the present disclosure provide a treatment device for skin treatment, which finely control the temperature of the deep skin by controlling the output energy per pulse within a treatment cycle, while maintaining a constant total output energy.

Embodiments of the present disclosure provide a treatment device, which controls the number and timing of coolings for a treatment to reduce skin irritation (pain) caused by RF energy irradiation.

According to an embodiment, a pain relief device for relieving pain through control of output energy for each pulse and timing of cooling includes an electrode part in contact with surface of skin, an energy supply part that supplies energy to the electrode part during a reference cycle, a cooling part, and a processor that controls the energy supply part and the cooling part.

The processor may separate the reference cycle into a plurality of pulse shot intervals and control the energy supply part to maintain constant output energy during all the separated pulse hot intervals, and control the cooling part to lower a temperature of the surface of the skin by a preset temperature in at least a portion between the separated pulse shot intervals.

The pain relief device may further include a temperature sensor that measures a temperature of a treated portion of the skin.

The processor may monitor a change in the temperature of the treated portion of the skin through the temperature sensor, and, when the temperature of the treated portion is higher than a preset threshold, control the cooling part to lower the temperature of the treated portion.

The electrode part may include a plurality of electrodes, and the processor may control the energy supply part to supply power to some or all of the plurality of electrodes during the reference cycle based on the change in the temperature of the treated portion of the skin.

The processor may separate the reference cycle into a first interval and a second interval based on a specified time point within the reference cycle, and control the energy supply part such that pulses with different energies are irradiated in the first interval and the second interval.

The processor may control the energy supply part such that a pulse with first energy is irradiated in the first interval, and control the energy supply part such that a pulse with second energy lower than the first energy is irradiated in the second interval.

The processor may control the energy supply part such that a pulse with first energy is irradiated in the first interval, and control the energy supply part such that a pulse with progressively lower energy from the first energy is irradiated in the second interval.

According to an embodiment, the pain relief device may further include a user input part that collect, from a patient, whether and when a pain has occurred, and the processor may receive, from a specific patient via the user input part, whether the pain has occurred and when the pain has occurred while irradiating pulses to the specific patient during the reference cycle, and calculate the specified time point based on time when the pain has occurred.

The processor may calculate a beginning of a specific pulse irradiated at the time the pain has occurred, as the specified time point.

The processor may determine intensities of pulses to be irradiated in the first interval and the second interval based on an intensity of the pulse irradiated at the time the pain occurred.

The processor may control the energy supply part such that an intensity of a pulse to be irradiated in the first interval is lower than a maximum intensity of the specific pulse irradiated at the time the pain occurred.

In addition, a computer program stored in a computer-readable recording medium may be further provided to execute the method for implementing the present disclosure.

In addition, a computer-readable recording medium for recording a computer program for executing the method for implementing the present disclosure may be further provided.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a diagram for describing an environment in which a device is applied, which relieves pain through control of output energy for each pulse and timing of cooling during surface electrode-based RF therapy according to the present disclosure;

FIG. 2 is a block diagram showing a configuration of a pain relief device that relieves pain through control of output energy for each pulse and timing of cooling during surface electrode-based RF therapy according to the present disclosure;

FIG. 3 is a diagram for describing a change in output energy when a plurality of pulse shots is irradiated to skin within a reference cycle according to the present disclosure; and

FIG. 4 is a diagram for describing a method for lowering a temperature of skin surface according to the present disclosure.

### DETAILED DESCRIPTION

Like reference numerals refer to like elements throughout the specification. The present disclosure does not describe all elements of the embodiments, and general content in the technical field to which the present disclosure pertains or overlapping content between embodiments are omitted. Terms such as “unit”, “module”, “member”, and “block” may be embodied as hardware or software. According to embodiments, a plurality of “unit”, “module”, “member”, and “block” may be implemented as a single component or a single “unit”, “module”, “member”, and “block” may include a plurality of components.

It will be understood that when an element is referred to as being “connected to” another element, it can be directly or indirectly connected to the other element, wherein the indirect connection includes “connection via a wireless communication network”.

Also, when a part “includes” or “comprises” an element, unless there is a particular description contrary thereto, the part may further include other elements, not excluding the other elements.

Throughout the specification, a certain member being located “on” another member includes not only a case where the certain member is in contact with another member, but also a case where another member exists between two members.

Terms such as first and second are used to distinguish one component from another component, and the components are not limited by the above-mentioned terms.

As used herein, singular forms may include plural forms as well unless the context clearly indicates otherwise.

In each step, identification symbols are used for convenience of description and the identification symbols do not describe the order of the steps and the steps may be performed in a different order from the described order unless the context clearly indicates a specific order.

Hereinafter, the operating principle and embodiments of the present disclosure will be described with reference to the attached drawings.

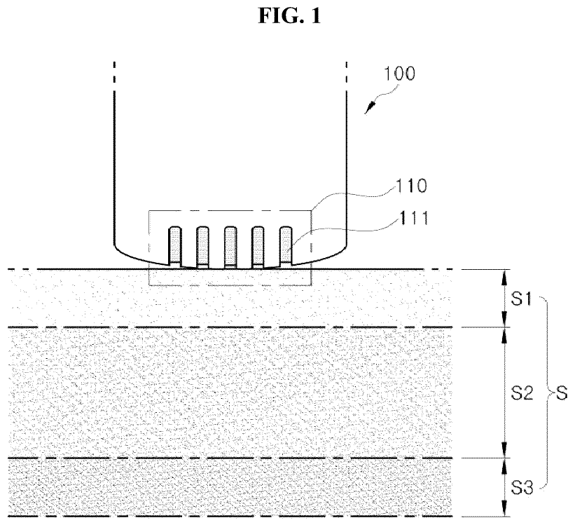

FIG. 1 is a diagram for describing an environment in which a device 100 (hereinafter referred to as a “pain relief device”) is applied, which relieves pain through control of output energy for each pulse and timing of cooling during surface electrode-based RF therapy according to the present disclosure.

The pain relief device 100 may be based on non-invasive and may output high frequency waves toward the inside of skin for treatment or cosmetic purposes. Here, the high frequency waves may have a predetermined frequency band based on RF (Radio Frequency).

The pain relief device 100 may output at least one high frequency of 6.78 mHZ, 13.56 mHZ, and 27.12 Mhz, but the embodiment is not limited thereto.

In an embodiment, when the pain relief device 100 irradiates RF energy at 6.78 mHZ, the pain relief device 100 may deliver energy to deep skin layers because the RF energy has a relatively low frequency. Therefore, the pain relief device 100 may be suitable for treating deep skin layers, and energy may be delivered to a wide area.

In an embodiment, when the pain relief device 100 irradiates RF energy at 13.56 mHZ, the pain relief device 100 may deliver RF energy to the middle layers of the skin because the RF energy has an intermediate frequency. The frequency may be effective in the case of treating the layers between the deep skin layer and the surface layer.

In an embodiment, when the pain relief device 100 irradiates RF energy at 27.12 mHZ, the pain relief device 100 may deliver energy intensively to the surface layer of the skin because the RF energy has a relatively high frequency. Due to the nature of high frequency, it is possible to deliver energy intensively to a narrow and specific area, which may be effective for precise treatment or cosmetic procedures in specific areas.

As descried above, as the frequency increases, energy may be concentrated in a narrower area, which may be advantageous for more precise treatment or procedures in specific areas. On the other hand, low frequencies may deliver energy to a wide area, which may be effective in treating deep skin layers or treating large areas. Accordingly, various frequencies may be selected depending on the purpose of treatment or procedure, a target area, a desired result and/or the like.

The pain relief device 100 may irradiate high-frequency pulse shots to the skin (S) by supplying power to an electrode part 110, which includes a plurality of (surface) electrodes disposed at positions in contact with the skin surface. Here, a gap may occur between the skin surface and the electrode (e.g., 111) due to the structure (e.g., including the housing) of the pain relief device 100.

The pain relief device 100 may deliver energy to not only the epidermis (S1) of the skin (S) but also the dermis (S2) of the skin (S), and use heat generated by the high frequency to remove damaged collagen, elastic fibers, or the like from the deep portion of the skin and promote new formation. However, it is difficult for the pain relief device 100 to reach the subcutaneous fat (S3) of the skin (S).

The pain relief device 100 according to the present disclosure does not supply a one-time, high-intensity power to the electrode part 110 at a reference cycle of treatment. The pain relief device 100 may divide the reference cycle into a plurality of intervals, reduce the power intensity to be relatively weak and then supply the power to the electrode part 110 at each interval. Accordingly, skin damage may be reduced, skin temperature does not rise rapidly, and skin pain may be relieved.

The reference cycle is a default unit time for an energy supply part 120 to irradiate RF energy to the skin through the electrode part 110, and may be set within a range from the minimum irradiation time to the maximum irradiation time of RF energy. The plurality of pulse shot intervals may be arranged sequentially within the reference cycle.

Here, the reference cycle may be set from 1 nanosecond to 1 minute, but the embodiment is not limited thereto. The energy output range per pulse shot may be a range of from 0.1 wATTS to 500 watts, but the embodiment is not limited thereto.

As described above, the pain relief device 100 may divide the reference cycle into several intervals and supply energy, thereby reducing skin damage rather than supplying strong energy to the skin all at once. Additionally, it is possible to prevent a rapid rise in skin temperature by controlling the energy intensity for each interval, thereby reducing skin pain.

Even though the pain relief device 100 according to the present disclosure irradiates the skin with pulse shots several times within the reference cycle of one treatment, the total amount of output energy used for the reference cycle of one treatment may be supplied in the same manner as in the case of supplying high power to the electrode part 110 within the reference cycle on a one-time basis. Accordingly, a rapid rise in skin temperature may be prevented while excellent skin treatment effects is being maintained.

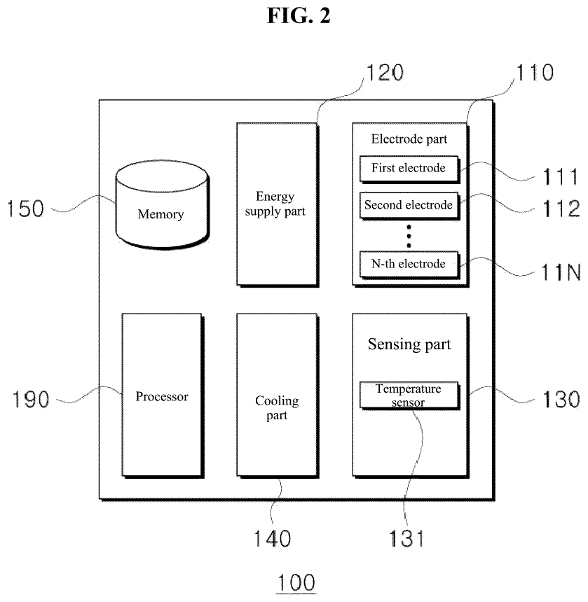

Hereinafter, the pain relief device 100 will be described in more detail with reference to FIG. 2.

FIG. 2 is a block diagram illustrating a configuration of the pain relief device 100 according to the present disclosure.

The pain relief device 100 may include the electrode part 110 including a plurality of electrodes 111 to 11N, the energy supply part 120 that applies power to the electrode part 110, a sensing part 130, a cooling part 140, a memory 150, and at least one processor 190. The components of the pain relief device 100 shown in FIG. 2 are not essential for implementing the pain relief device 100 according to the present disclosure, so the pain relief device 100 described herein may include more or fewer components than the components listed above.

Among the above components, the electrode part 110 including the plurality of electrodes 111 to 11N is located on the surface of skin, and therefore, is referred to as a surface electrode. The electrode part 110 may be located inside the structure (e.g., housing) of the pain relief device 100.

In an embodiment, the electrode part 110 may include one or more electrodes, and the arrangement shape of the electrodes may be implemented in a circle, polygon (e.g., triangle, square, or the like), or the like and the arrangement shape may be an arrangement shape in which a plurality of electrodes are symmetrically arranged according to the shape of arrangement of the electrodes, but the present disclosure is not limited thereto. Furthermore, the electrodes may be implemented in various shapes, such as round, square, hexagonal, or the like and the size and arrangement of the electrodes may be adjusted according to the treatment area. Additionally, conductive gels or solutions may be used to improve skin contact.

The energy supply part 120 may supply energy to the electrode part 110 during the reference cycle. Here, the reference cycle may be a time period when treatment or procedure is performed, and may be a time period corresponding to a single treatment or procedure. A plurality of reference cycles may be included within a single treatment or procedure, but the embodiment is not limited thereto.

In one embodiment, the energy supply part 120 may simultaneously supply energy to all of the plurality of electrodes 111 to 11N.

In another embodiment, the energy supply part 120 may supply energy to a specific electrode. When the temperature of a skin area in contact with the specific electrode is higher than a preset value, an increase in skin temperature may be controlled by supplying energy to another electrode while avoiding the skin area. Additionally, the energy supply part 120 may supply energy to the electrodes sequentially (or alternately) according to the specified order of the electrodes.

The present disclosure may provide various variations of energy supply methods. For example, the energy supply may be performed with continuous waves, pulsed waves, or a combination thereof, and various energy delivery patterns may be implemented through frequency modulation or amplitude modulation.

The sensing part 130 is a module that detects environmental information related to an external environment and may typically include a temperature sensor 131. The temperature sensor 131 may detect the temperature of a treated portion when a skin treatment is performed using the pain relief device 100.

In an embodiment, the temperature sensor 131 may monitor the entire skin area of the treated portion and sense an area or portion where the temperature exceeds a predetermined threshold, but the present disclosure is not limited thereto.

The cooling part 140 is a module for lowering the skin temperature of the entire area or a portion of the treated portion, and may lower the temperature of the skin surface with cooling gas, but the embodiment is not limited thereto.

The memory 150 may store data supporting various functions of the pain relief device 100 and programs for operation of the processor, may store input/output data, and may store a number of applications (application programs or applications) running on the pain relief device 100, and data and instructions for operation of the pain relief device 100. The memory 150 may store one or more instructions and may include various deep learning-based models.

The memory 150 may include at least one type of storage medium among a flash memory type, a hard disk type, a solid state disk type, a Silicon Disk Drive type (SDD) type, a multimedia card micro type, a card type memory (e.g., SD or XD memory), random access memory (RAM), static random access memory (SRAM), read-only memory (ROM), electrically erasable programmable read-only memory (EEPROM), programmable read-only memory (PROM), magnetic memory, magnetic disk, and optical disk. Additionally, the memory 150 may be a database that is separate from the pain relief device 100 but connected wired or wirelessly, and may be implemented as a database system.

The processor 190 may include at least one core and may execute instructions stored in the memory 150. The processor 190 may be implemented with a memory that stores data for an algorithm or program reproducing the algorithm for controlling the operations of components within the pain relief device 100, and at least one processor (not shown) that performs the aforementioned operations using the data stored in the memory. In this case, the memory and the processor may be implemented as separate chips. Additionally, the processor 190 may control the components of the pain relief device 100.

At least one component may be added or deleted in response to the performance of the components shown in FIG. 2. Additionally, it will be easily understood by those skilled in the art that the mutual positions of the components may be changed in response to the performance or structure of a system.

Meanwhile, each of components shown in FIG. 2 may refer to a software and/or hardware component such as Field Programmable Gate Array (FPGA) and Application Specific Integrated Circuit (ASIC).

From a clinical perspective, the above-described pain relief device 100 may present a new method of alleviating pain during RF therapy. The RF therapy may deliver energy to the deep portion of skin to produce a therapeutic effect, but the process of RF therapy may be painful as the temperature of the skin surface increases.

The pain relief device 100 may control the intensity of energy provided for each pulse shot to minimize an increase in temperature on skin surface while delivering sufficient energy to the deep portion of skin. Accordingly, the therapeutic effect may be maintained while reducing a patient's pain.

Additionally, from a technical perspective, the pain relief device 100 may irradiate a plurality of pulse shots within one cycle using technology based on dielectric RF electrodes. In the process, the pain relief device 100 may control the intensity of energy provided for each pulse shot, which may deliver sufficient energy to the deep skin while minimizing the increase in temperature on the skin surface.

The pain relief device 100 may constantly maintain total energy provided during the reference cycle through energy control, thereby ensuring the same treatment effect as the conventional RF therapy.

A detailed algorithm for a process of controlling the processor according to the present disclosure may be as follows:

1. Set an initial parameter of at least one of an energy level and a pulse interval.

2. Supply energy for a first pulse shot interval.

3. Monitor a skin temperature via a temperature sensor.

4. Activate a cooling system when, as a result of the monitoring, the skin temperature exceeds a preset temperature.

5. Adjust an energy level for the next pulse shot interval.

6. Receive and process feedback on user pain.

7. Repeatedly perform the operations of steps 2 to 6 above until the reference cycle is complete.

8. Evaluate the overall treatment effectiveness and optimize the parameter for the next session.

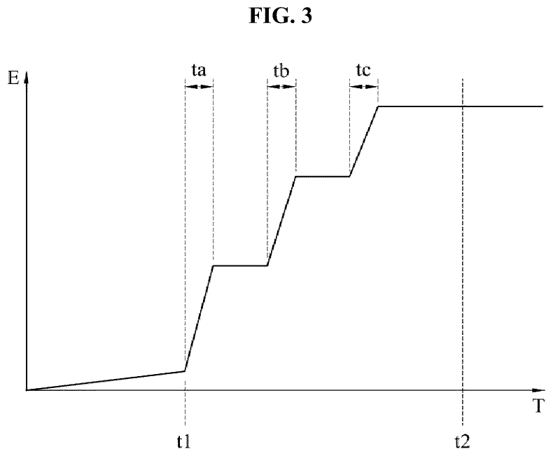

FIG. 3 is a diagram for describing a change in output energy when a plurality of pulse shots is irradiated to skin within a reference cycle (t1 to t2) according to the present disclosure.

The processor 190 may separate the reference cycle (t1 to t2) into a plurality of pulse shot intervals (including ta to tc). The plurality of pulse shot intervals are illustratively shown as three intervals, but more or fewer intervals may be included depending on an embodiment.

It should be noted that the processor 190 may control the energy supply part 120 to constantly maintain the output energy of all of separated pulse shot intervals (reference cycle). In this case, when the output energy is the same, the same skin treatment effect may be expected even through the pulse shot is irradiated multiple times.

As described above, the pain relief device 100 may present a new method of effectively managing the increase in temperature of skin surface during a RF therapy process. The pain relief device 100 may irradiate a plurality of pulse shots within a reference cycle while maintaining a constant output energy during the reference cycle to ensure therapeutic effectiveness to reduce the increase in skin temperature to reduce pain, and increase patient comfort, without using a simplistic method of managing the increase in temperature of the skin surface by providing cooling between pulses. Accordingly, the temperature of the skin surface may be managed more effectively, and in particular, by controlling the energy of each pulse shot, sufficient energy may be delivered to the deep portion of skin while minimizing the increase in temperature of the skin surface.

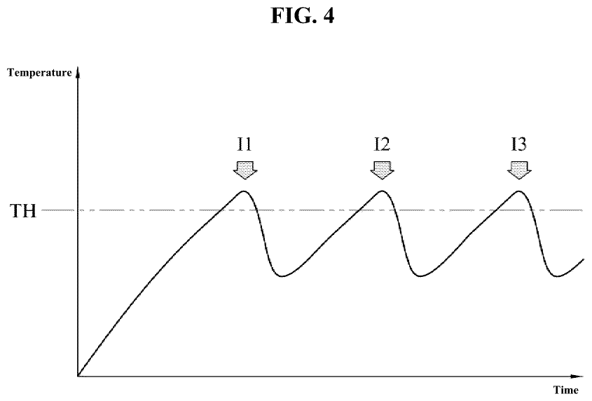

FIG. 4 is a diagram for describing a method for lowering a temperature of skin surface according to the present disclosure.

The processor 190 may perform skin treatment through a plurality of pulse shots within a reference cycle, and control the cooling part 140 to lower the temperature of the surface of skin by a preset temperature in at least part between separated pulse shot intervals.

The processor 190 may lower the temperature of skin through the cooling part 140 by controlling the number of and timing of coolings between the plurality of pulse shots.

Here, the number of coolings may be one or more, and the timing of cooling may be before or after high-frequency output, and the cooling may also be performed simultaneously with high-frequency output.

The pain relief device 100 according to the present disclosure may present a new method of increasing pain relief effect by controlling the number and timing of coolings during the RF therapy process. The pain relief device 100 may more effectively control the increase in the temperature of the skin's surface without using the conventional method of performing cooling at regular intervals.

The pain relief device 100 may prevent excessive increase in temperature of the skin surface by controlling the number and timing at which cooling is provided, thereby relieving pain more effectively.

In an embodiment, the pain relief device 100 may include the temperature sensor 131 that measures the temperature of the treated portion of the skin. The pain relief device 100 may monitor a change in temperature of the treated portion of the skin through the temperature sensor 131, and when the temperature of the treated portion is higher than a preset threshold (TH), perform control such that the cooling part 140 lowers the temperature of the treated portion between pulse shot intervals.

In an embodiment, the pain relief device 100 may supply power to all or individual electrodes included in the electrode part 110. In this case, the processor 190 may control the energy supply part 120 to supply power to some or all of the plurality of electrodes during a reference cycle based on a change in the temperature of the treated portion of the skin.

For example, the processor 190 may irradiate a pulse shot via the first electrode to a specific area of skin only, and when the temperature of the specific area is raised by a preset temperature due to the first electrode, irradiate a pulse shot to a different area using the second electrode. In this case, the processor 190 may control the cooling part 140 to reduce the temperature of the skin area raised due to the first electrode prior to irradiation of the pulse shot via the second electrode, but the present disclosure is not limited thereto.

In an embodiment, the processor 190 may monitor the change in temperature of the entire or portion of the treated portion in real time using the temperature sensor 131 and, when the change in temperature change of the entire or portion of the treated portion is greater than a preset threshold temperature, control the cooling part 140 to perform cooling on the skin, while overriding the scheduled cooling application.

Further, the pain relief device 100 according to the present disclosure may irradiate a plurality of pulses when irradiating a single shot, and the plurality of pulses may be separated by time intervals. For example, a single shot may include two pulses, and the two pulses may be set to be irradiated in a first interval and a second interval, with the earlier interval being the first interval and the later interval being the second interval based on a specified time point.

The pain relief device 100 according to the present disclosure may reduce a patient's pain by utilizing such a plurality of pulses.

The processor 190 may receive input from the patient as to a specific time point at which the patient felt pain after irradiating a single shot including a plurality of pulses to the patient for a preset time period. Through this, the processor 190 may calculate a pulse at which a specific patient felt pain, and may calculate the specific time point based on the calculated result.

To this end, the pain relief device 100 according to the present disclosure may further include a user input part configured to collect from a patient whether and when a pain has occurred. The patient may input the occurrence of pain via the user input part when pain is felt during treatment during a reference cycle including plurality of pulses. Therefore, the present disclosure may collect whether or when a user felt a pain.

In one embodiment, the user input part may include a mechanical button mounted on the exterior of the pain relief device 100 to be activated by pressing or touching of the user, a capacitive, pressure sensitive, or ultrasonic touchscreen to be activated by direct contact with a part of the user's body, and a voice recognition module configured to recognize the user's voice to extract pain-related information, and may transmit pain information collected via at least one of the mechanical button, the touchscreen, and the voice recognition module to the processor 190.

In one embodiment, the user input part may be implemented in a method of pressing/touching a mechanical button, a capacitive/pressure/ultrasonic sensing method for a touchscreen, a voice recognition function, an information extraction function, or/and the like, but the present disclosure is not limited thereto. The processor 190 may calculate the specified time point at which the first and second intervals are separated based on a time point at which the pain occurred.

In one embodiment, the processor 190 may calculate the beginning of the pulse in which the patient felt pain as the specified time point. That is, the beginning of the pulse that caused the patient's pain may be set as the reference time point that separates the first and second intervals.

The processor 190 may then perform separation into the pulses in the first interval and the pulses in the second interval based on the specified time point, and perform control that pulses with different energies are irradiated in different intervals.

By arranging at least one pulse in each of the first interval and the second interval, the processor 190 may customize the energy irradiated during the reference cycle to reduce the patient's pain. Specifically, the processor 190 may control the energy of the pulses irradiated to minimize the patient's pain by irradiating high-energy pulses in the early part of the energy irradiation and controlling the energy of the pulses irradiated in the later part of the energy irradiation, in which pain is primarily caused.

To this end, the pulses irradiated in the first interval may have higher energy than the pulses irradiated in the second interval.

When the second interval has been reached, the processor 190 may irradiate pulses with lower energy than that of the first interval, or irradiate pulses with progressively lower energy from that of the first interval.

In one embodiment, the processor 190 may control the energy supply part 120 such that a pulse with first energy is irradiated in the first interval, and may control the energy supply part 120 such that a pulse with second energy lower than the first energy is irradiated in the second interval.

In one embodiment, the processor 190 may control the energy supply part 120 such that a pulse with first energy is irradiated in the first interval, and may control the energy supply part 120 such that a pulse with progressively lower energy from the first energy is irradiated in the second interval.

Further, the pain relief device 100 according to the present disclosure may determine the intensities of pulses to be irradiated in the first interval and the second interval based on the intensity of the pulses irradiated at the time the pain occurred.

In one embodiment, the processor 190 may control the energy supply part 120 such that the intensity of the pulses irradiated in the first interval is lower than the maximum intensity of a specific pulse irradiated at the time the pain occurred.

Through this, the present disclosure may minimize the likelihood of patient experiencing pain by irradiating pulses with lower intensity than that of the energy irradiated at the time the patient experienced pain in a distributed manner, during time intervals of the reference cycle.

Furthermore, the present disclosure may maintain therapeutic effectiveness with reduced skin damage and reduced pain. Furthermore, the skin temperature may be lowered, reducing skin damage.

Furthermore, the present disclosure may effectively suppress the rapid increase in temperature of the skin surface during RF therapy by controlling the pulse energy and cooling. By controlling the energy of each pulse, the instantaneous thermal energy delivered to the skin may be regulated, thereby preventing excessive increase in temperature of skin, which may cause pain.

Furthermore, by providing adequate cooling between pulses, the present disclosure may continuously cool the heat on the surface of the skin, thereby suppressing the increase in temperature of skin.

Furthermore, the present disclosure may control pulse energy and cooling to allow RF energy to be delivered to the dermal layer while maintaining the temperature of the skin's epidermis and dermis at a safe level, thereby significantly reducing the risk of pain and skin damage while maintaining the therapeutic benefits of conventional RF therapy.

In addition, the present disclosure may control the intensity of RF energy and the degree of cooling according to skin characteristics, enabling treatment optimized for various skin types and individual differences in pain sensitivity.

Through the above effects of the present disclosure, it may be expected that patient compliance and satisfaction with RF therapy is increased, and practitioners is able to provide more effective and safer RF therapy.

The present disclosure may further adjust irradiation time and intensity of the radiofrequency energy based on the real-time measured skin impedance. In this case, the present disclosure may estimate moisture content of the skin based on the skin impedance measurement, and may adjust radiofrequency irradiation energy accordingly.

The present disclosure may automatically activate cooling function when a skin temperature is higher than a predetermined target temperature. In this case, the present disclosure can also notify a user when the cooling function is activated.

The present disclosure can also detect any abnormal conditions that may occur during skin care in real time and alert the user.

The present disclosure may combine skin temperature measurement function and skin impedance measurement function to determine optimal radiofrequency energy irradiation conditions. At this time, the present disclosure may allow the user to select various radiofrequency energy irradiation patterns via a user interface. Furthermore, the present disclosure allows the user to preset different radiofrequency energy irradiation patterns for different skin types. At this time, the present disclosure allows the user to save the preset radiofrequency energy irradiation patterns and quickly recall them when needed. Furthermore, the present disclosure may optimize the user interface so that the user can easily select a radiofrequency energy irradiation pattern that suits their skin type.

The present disclosure may analyze the effect of the irradiated radiofrequency energy in real time and provide feedback to the user. In this case, the present disclosure may visually display the results of analyzing the effect of the irradiated radiofrequency energy to the user.

The present disclosure may provide a cooling pattern adapted to the skin based on the moisture content of the skin. In this case, the present disclosure may automatically set optimal cooling conditions based on the moisture content of the skin. Further, the present disclosure may automatically adjust the irradiation time and intensity of the radiofrequency energy based on the moisture content of the skin.

The present disclosure may change the irradiation pattern of the radiofrequency energy selected by the user. In this case, the present disclosure may analyze and adjust the response of the skin according to the irradiation pattern of the radiofrequency energy selected by the user.

The present disclosure may automatically adjust the skin care program based on the user's skin condition. Based on the skin temperature and impedance measurement results, the present disclosure may recommend a customized radiofrequency irradiation protocol for a specific skin problem. In some embodiments, the disclosure may integrate the results of the skin temperature and impedance measurements to more accurately determine the condition of the skin.

As described above, the present disclosure provides the following key features and advantages.

First, the reference cycle may be divided into a plurality of pulse shot intervals to supply the energy in a distributed manner. Second, the energy of each pulse shot interval may be individually controlled to minimize the increase in skin surface temperature. Third, the timing and number of coolings may be dynamically controlled for effective temperature management. Fourth, an energy adjustment function that reflects the user's pain feedback may be provided in real time. Fifth, customized treatment for various skin types and individual pain sensitivity may be provided.

Further, the present disclosure may be implemented using ultrasonic waves, lasers, and/or a combination thereof, in addition to RF energy. For example, RF energy and ultrasonic waves may be applied alternately, or lasers may be used adjunctively during RF energy delivery. Cooling may also be implemented in various ways, such as contact cooling, jet cooling, or cooling using thermoelectric elements.

On the other hand, the disclosed embodiments may be implemented in the form of a recording medium storing instructions executable by a computer. The instructions may be stored in the form of program codes, which, when executed by the processor, may generate program modules to perform the operations of the disclosed embodiments. The recording medium may be implemented as a computer-readable recording medium.

The computer-readable recording medium may include all types of recording media storing instructions capable of being decoded by a computer. For example, the computer-readable recording medium may include read only memory (ROM), random access memory (RAM), magnetic tape, magnetic disc, flash memory, optical data storage, and the like.

The embodiments of the disclosure have been described above with reference to the accompanying drawings. A person skilled in the art to which this disclosure pertains will understand that the present disclosure may be practiced in forms different from the disclosed embodiments without changing the technical idea or essential features of the present disclosure. The disclosed embodiments are illustrative and should not be construed as limiting.

While the present disclosure has been described with reference to embodiments, it will be apparent to those skilled in the art that various changes and modifications may be made without departing from the spirit and scope of the present disclosure. Therefore, it should be understood that the above embodiments are not limiting, but illustrative.

What is claimed is:

1. A pain relief device for relieving pain through control of output energy for each pulse and timing of cooling, the pain relief device comprising:
an electrode part in contact with surface of skin;
an energy supply part configured to supply energy to the electrode part during a reference cycle;
a cooling part; and
a processor configured to control the energy supply part and the cooling part,
wherein the processor is configured to:
separate the reference cycle into a plurality of pulse shot intervals and control the energy supply part to maintain constant output energy during all the separated pulse hot intervals; and
control the cooling part to lower a temperature of the surface of the skin by a preset temperature in at least a portion between the separated pulse shot intervals,
wherein the reference cycle is a default unit time for the energy supply part to irradiate RF energy to the skin through the electrode part, and is set within a range from a minimum irradiation time to a maximum irradiation time of the RF energy, and
wherein the plurality of pulse shot intervals are arranged sequentially within the reference cycle.

2. The pain relief device of claim 1, further comprising:
a temperature sensor configured to measure a temperature of a treated portion of the skin,
wherein the processor is configured to:
monitor a change in the temperature of the treated portion of the skin through the temperature sensor; and
when the temperature of the treated portion is higher than a preset threshold, control the cooling part to lower the temperature of the treated portion.

3. The pain relief device of claim 2, wherein the electrode part includes a plurality of electrodes,
wherein the processor is configured to control the energy supply part to supply power to some or all of the plurality of electrodes during the reference cycle based on the change in the temperature of the treated portion of the skin.

4. The pain relief device of claim 1, wherein the processor is configured to:
separate the reference cycle into a first interval and a second interval based on a specified time point within the reference cycle; and
control the energy supply part such that pulses with different energies are irradiated in the first interval and the second interval.

5. The pain relief device of claim 4, wherein the processor is configured to:
control the energy supply part such that a pulse with first energy is irradiated in the first interval; and
control the energy supply part such that a pulse with second energy lower than the first energy is irradiated in the second interval.

6. The pain relief device of claim 4, wherein the processor is configured to:
control the energy supply part such that a pulse with first energy is irradiated in the first interval; and
control the energy supply part such that a pulse with progressively lower energy from the first energy is irradiated in the second interval.

7. The pain relief device of claim 4, further comprising:
a user input part configured to collect, from a patient, whether and when a pain has occurred,
wherein the processor is configured to receive, from a specific patient via the user input part, whether the pain has occurred and when the pain has occurred while irradiating pulses to the specific patient during the reference cycle, and calculate the specified time point based on time when the pain has occurred.

8. The pain relief device of claim 7, wherein the user input part includes:
a mechanical button mounted on exterior of the pain relief device to be activated by pressing or touching of a user;
one of a capacitive touchscreen, a pressure sensitive touchscreen, or an ultrasonic touchscreen to be activated by direct contact with a part of the user's body; and
a voice recognition module configured to recognize a user's voice to extract pain-related information, and
wherein the user input part is configured to transmit pain information collected via at least one of the mechanical button, the touchscreen, and the voice recognition module to the processor.

9. The pain relief device of claim 7, wherein the processor is configured to calculate a beginning of a specific pulse irradiated at the time the pain has occurred, as the specified time point.

10. The pain relief device of claim 9, wherein the processor is configured to:
determine intensities of pulses to be irradiated in the first interval and the second interval based on an intensity of the pulse irradiated at the time the pain occurred, and
control the energy supply part such that an intensity of a pulse to be irradiated in the first interval is lower than a maximum intensity of the specific pulse irradiated at the time the pain occurred.
